(19) United States
(12) Patent Application Publication
(10) Pub. No.: US 2025/0136567 A1
(43) Pub. Date: May 1, 2025

(54) TOFA ANALOGS AND SEBUM REDUCING PREPARATIONS CONTAINING SAME

(71) Applicant: BEIERSDORF AG, Hamburg (DE)

(72) Inventors: Judith SEIDEL, Berlin (DE); Joern Hendrik REUTER, Henstedt Ulzburg (DE); Ahmed KAMAL, Hamburg (DE); Annika SIMMERING, Hamburg (DE); Heiko MIELKE, Neu Wulmstorf (DE)

(21) Appl. No.: 18/835,204

(22) PCT Filed: Jan. 16, 2023
(86) PCT No.: PCT/EP2023/050861

§ 371 Date: Aug. 1, 2024

(30) Foreign Application Priority Data
Feb. 8, 2022 (DE) 10 2022 201 276.3

(51) Int. Cl. C07D 307/68; A61K 8/49; A61Q 19/00; C07D 233/90; C07D 263/34; C07D 277/56

(52) U.S. Cl. CPC C07D 307/68; A61K 8/49; A61K 8/4946; A61K 8/4973; A61Q 19/008; C07D 233/90; C07D 263/34; C07D 277/56

(57) ABSTRACT

The invention relates to compounds of general formula (1).

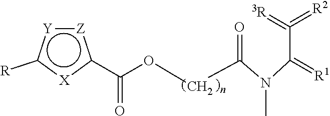

The present invention relates to novel substances and uses thereof as active ingredients and preparations containing such active ingredients which are effective against greasy and/or impure skin, and mild forms of acne and/or in the case of greasy hair.

Seborrhea is a predisposition to increased function of the sebaceous glands. Both scalp and facial skin appear greasy. The composition of seborrheic sebum is altered compared to normal sebum. Three stages of development of seborrhea are distinguished:
1. Simple seborrhea: mild cases, greasy after 8 days.
2. Oily seborrhea: already greasy after 2-3 days.
3. Irreversible form: no longer reversible. Seborrhea where the hair looks like it has been bathed in grease after just one day.

The excessive secretion of the sebaceous glands can, inter alia, be triggered by androgenetic disorders (male sex hormone disorder) and has a detrimental esthetic effect on the overall appearance of the hair. This disorder can also be the cause of hair loss that occurs. The precursor in each case is the seborrhoeic condition of the scalp. Vegetative disorders and improper care can worsen the appearance of the skin and also the condition of the hair. Even with seborrhea, the hair itself can be dry due to disturbances in keratin formation. Dry, weakened hair is often caused by external stress such as sun or chemical treatments. Excessively hot blow-drying or not properly caring for weakened hair can lead to damage.

The causes of greasy hair lie in the human body and are hormonal. Each hair has its own sebaceous gland, which produces grease (also called sebum). Sebum production is hormonally controlled, and over-or underproduction can occur, depending on the hormone sensitivity of the sebaceous gland. The sebum itself has the function of keeping the scalp supple. It passes from the sebaceous gland to the scalp and only later the hair root. There it is normally absorbed by the hair shaft and remains invisible. When sebum is overproduced, the hair shaft is no longer able to absorb it. It becomes visible as a greasy film on the hair. The result is stringy hair that is shiny with grease.

Due to the fact that sebaceous gland production is dependent on the hormone balance, the problem of greasy hair cannot be solved fundamentally since the sebaceous glands continually produce grease. Consistent care and high-quality skin care products are still the best way to combat greasy hair.

Unfortunately, greasy hair has very troublesome effects. The hair becomes stringy again just a short time after washing and the hairstyle does not hold.

Contrary to popular belief, it is only a rumor that the hair becomes greasy more quickly as a result of excessively frequent washing. Mild shampoos for greasy hair ensure that excess grease is removed. Hair and scalp are supplied with sufficient moisture and balance the overproduction of the sebaceous glands.

Greasy hair and dandruff are among the most common hair problems. These abnormalities can be attributed to a disorder in the activity of the sebaceous glands. If the sebaceous glands are hyperactive, the term used is seborrhea. Two forms can be distinguished here: the oily form (seborrhea oleosa) and the dry form (seborrhea sicca).

### Seborrhea Oleosa

This involves hyperactivity of the sebaceous glands, where the sebaceous glands produce too much, and too oily, sebum. The skin therefore has a greasy sheen, and the hair is greasy and stringy again right down to the tips just 2 to 3 days after washing.

### Seborrhea Sicca

This can likewise be attributed to hyperactivity of the sebaceous glands, but the skin sebum is drier and has a firmer consistency. With the small flakes of the epidermis, it forms large, easily friable sebum flakes. The scalp has a waxy sheen, the hair becomes greasy only at the roots, and the lengths and particularly the tips are dry and even brittle.

The treatment of seborrhea includes, first of all, regular and thorough washing of the head with special shampoos, which can be carried out as often as seems necessary. The washing should be combined with massage in the connective tissue because this empties the sebaceous glands more, which delays the regreasing.

In the case of impure skin and mild forms of acne, in addition to increased sebum production, other influences such as secondary bacterial infections are also of etiological importance. One of the most important microorganisms associated with impure skin is Propionibacterium acnes.

Impure skin and/or comedones adversely affect the well-being of those affected, even in mild cases. Since practically every adolescent is affected by impure skin to some degree, for many people there is a need to remedy this condition. Since a major factor here is the excessive production of sebum, in many cases it is possible to improve the condition of the skin just through the use of sebum-reducing active ingredients.

It was thus the object of the present invention to find preparations that are effective against greasy and/or impure skin, mild forms of acne and against greasy hair.

The prior art already discloses some attempts to solve this problem, but they do not disclose all the advantages of the development presented herein.

WO 2011/005660 discloses analogs of 5-(tetradecyloxy)-2-furancarboxylic acid (TOFA) and their antiseborrheic effect.

Document WO 2018/022797 discloses analogs of 5-(tetradecyloxy)-2-furancarboxylic acid (TOFA) and their effect against acne vulgaris.

Document WO 2019/115405 discloses analogs of pyrrole derivatives and their antiseborrheic effect.

However, it has surprisingly been found, and therein lies the achievement of the object, that novel compounds of the general formula (1)

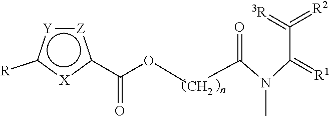

in which R=n-tetradecyl,
n=a number from 1 to 5,
X=O, N, N—H or S,
Y and Z independently are C—H, N, N—H, O or S, with the groups being selected such that the five-membered ring has Hückel-aromatic character,
that a double bond cannot simultaneously originate from R² and R³,
and R¹, R² and R³ independently are —H, an oxo group, C₁-C₂₄ alkyl (linear and branched), —C₁-C₂₄ alkenyl (linear and branched), —C₁-C₈ cycloalkyl, —C₁-C₈ cycloalkyl-alkylhydroxy, —C₁-C₂₄ alkyl (linear and branched) substituted by one or more hydroxyl groups and/or by one or more primary, secondary and/or tertiary amino groups and/or —C₁-C₂₄ alkyl (linear and branched) substituted with one or more optionally esterified carboxyl groups, —C₁-C₂₄ alkylaryl-alkylhydroxy (linear and branched), —C₁-C₂₄ alkylheteroaryl (linear and branched), —C₁-C₂₄ alkyl-O—C₁-C₂₄ alkyl (linear and branched), —C₁-C₂₄ alkylmorpholino, —C₁-C₂₄ alkylpiperidino, —C₁-C₂₄ alkylpiperazino, —C₁-C₂₄ alkylpiperazino-N-alkyl, oxo groups (═O)
with the proviso that at most one of the groups R¹, R² and R³ represents an H atom,
and medicinal or cosmetic topical preparations containing one or more such compounds at an effective concentration,
and the cosmetic use of one or more such compounds for reducing sebum production in human skin,
and the use of topical preparations containing one or more such compounds for the treatment of greasy and/or impure skin and/or mild forms of acne and/or greasy hair and/or greasy scalp and/or seborrhea, remedy the disadvantages of the prior art.

The uses according to the invention follow a similar principle, since the sebaceous glands on the scalp and on the face have an identical structure and the sebum production proceeds according to the same mechanism. The application of the sebum-reducing active ingredients on the scalp region thus makes it possible to reduce sebum production and therefore prevent the cosmetically undesirable condition of greasy hair.

Preparations that are particularly advantageous according to the invention are those that are characterized in that one or more compounds of the general formula (1), advantageously in a water and/or oil phase, are present at concentrations of 0.0001-40.00% by weight, preferably 0.005-20.00% by weight, particularly preferably 0.001-6.00% by weight, in each case based on the total weight of the composition.

Preferred compounds of formula (1) are:

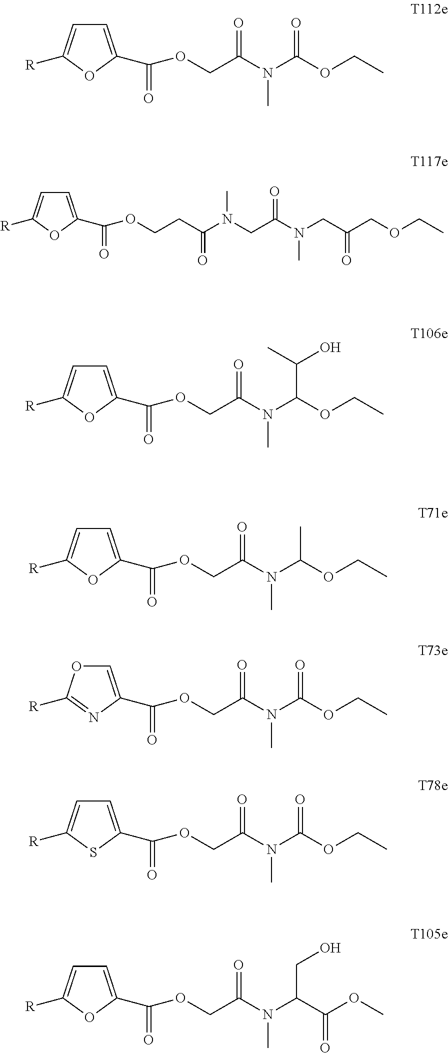

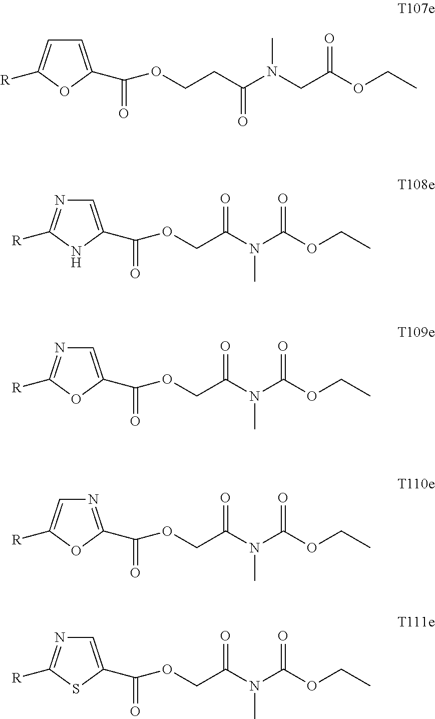

### Synthesis of TOFA (3)

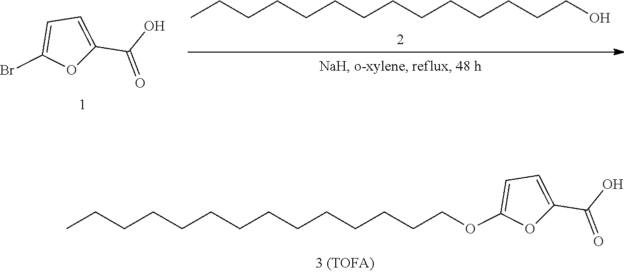

### Compound 3 (TOFA)

NaH (60% in mineral oil, 7.85 g, 196.3 mM) was added at ambient temperature to a suspension of alcohol (2) (25.3 g, 117.8 mM) in 400 ml of o-xylene and the mixture was stirred for 2 hours under reflux cooling. The mixture was cooled down to approx. 70° C., and acid (1) was added in solid form in one portion (15.0 g, 78.5 mM). The mixture was stirred for 48 h under reflux cooling and cooled to ambient temperature. The mixture was quenched with aqueous KHSO₄, extracted with EtOAc, the organic layer was additionally washed with H₂O, dried with Na₂SO₄ and evaporated. The residue was triturated with hexane, which gave Compound (3) (7.1 g, 28%). The synthesis was repeated several times in order to obtain the required amount of TOFA (approx. 100 g).

### ZE33-0100 (BDF: T71e)

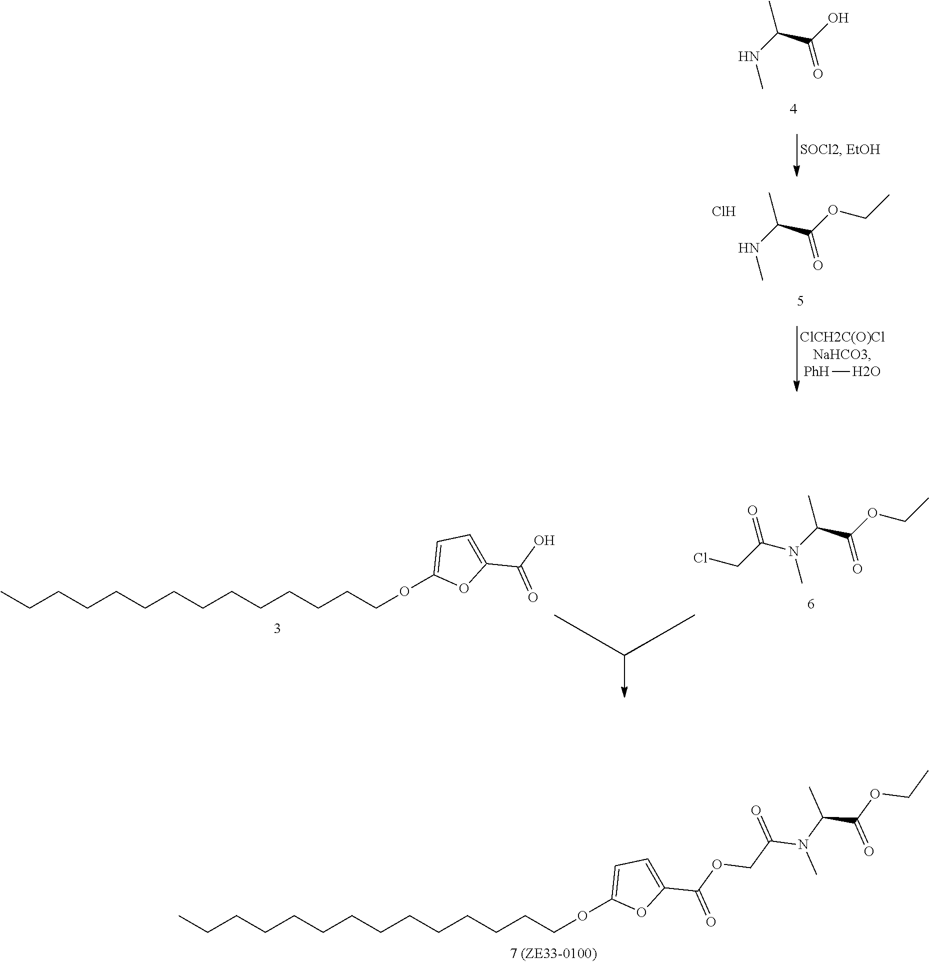

### Compound (5)

SOCl₂ (34.6 g, 300 mM) was added dropwise at 0° C. to a solution of Compound (4) (15.0 g, 150 mM) in EtOH (100 ml). The resulting mixture was heated for 12 h under reflux cooling. The mixture was subsequently left to stand until it had cooled to room temperature and was then concentrated under reduced pressure. After removal of the solvent, the residue was poured into aqueous NaHCO₃. The aqueous solution was extracted with DCM, and the extract was washed successively with aqueous NaHCO₃, H₂O, sodium chloride solution and dried over Na₂SO₄. Compound (5) (18.0 g, 94%) is obtained after concentration under reduced pressure. The material is used in the next step without purification.

### Compound (6)

Compound (5) (12.4 g, 150 mM) was dissolved in 160 ml of water. The solution was cooled in an ice bath and admixed with NaHCO₃ (14.3 g, 170.2 mM) in several steps. The reaction mixture was mixed dropwise with a solution (7.5 g, 66.6 mM) of chloroacetyl chloride in 140 ml of toluene, then stirred vigorously at 20-25° C. for 3 h. After a phase separation, the aqueous phase was extracted with toluene. The combined organic phases were freed of the solvent, Compound (6) was obtained as a colorless oil (8.46 g, 55%).

### Compound (7) (ZE33-0100) (BDF: T71e)

Compound (6) (1.2 eq., 11.0 g, 56.0 mM) and triethylamine (2 eq., 9.5 ml, 94.01) were added mM) at room temperature to a solution of TOFA (3) (15.4 g, 47.0 mM) in DMF (150 ml). After the addition, the mixture was stirred at 50° C. for 18 h and then the mixture was diluted with ether (250 ml) and washed with water (2×50 ml). The two layers were separated and the aqueous phase was extracted with dichloromethane (2×50 ml). The organic layers were combined, dried over anhydrous MgSO₄ and evaporated under reduced pressure to give an orange-colored oil. The resulting material was purified by silica gel column chromatography (with elution with 10-20% EtOAc/DCM) to produce Compound (7) (ZE33-0100) as white crystals (9.0 g, 39%).

### ZE33-0101 (BDF: T105e)

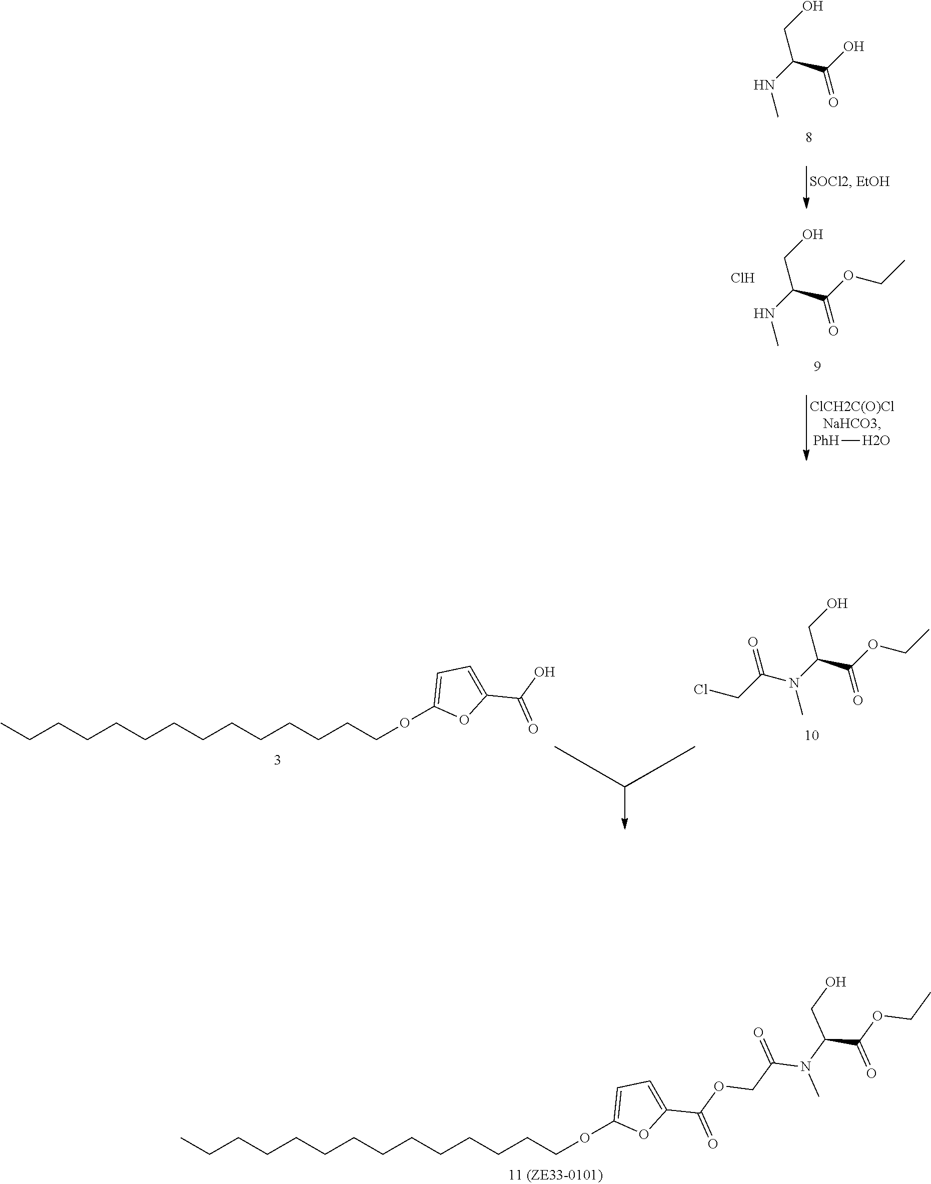

### Compound (9)

SOCl₂ (7.6 g, 64.5 mM) was added dropwise at 0° C. to a solution of Compound (8) (2.0 g, 12.9 mM) in EtOH (20 ml). The resulting mixture was heated for 12 h under reflux cooling. The mixture was subsequently left to stand at room temperature to cool and then concentrated under reduced pressure. After removal of the solvent, the residue was poured into aqueous NaHCO₃. The aqueous solution was extracted with DCM, and the extract was washed successively with aqueous NaHCO₃, H₂O, sodium chloride solution and dried over Na₂SO₄. Concentration under reduced pressure gives Compound (9) (2.2 g, 93%) as a colorless powder, which was used directly for the next reaction.

### Compound (10)

Compound (9) (2.2 g, 12.5 mM) was dissolved in 40 ml of water. The solution was cooled using an ice bath and admixed with (2.6 g, 31.3 mM) NaHCO₃ in several steps. The reaction mixture was admixed dropwise with a solution (1.3 g, 11.9 mM) of chloroacetyl chloride in 25 ml of toluene, then stirred vigorously at 20-25° C. for 3 h. After a phase separation, the aqueous phase was extracted with toluene. The combined organic phases were freed of the solvent, and Compound (10) was obtained as a colorless oil (2.5 g, 90%).

### Compound (11) (ZE33-0101) (BDF: T105e)

4 (1.1 eq., 7.6 g, 33.9 mM) and triethylamine (2 eq., 6.3 ml, 62.6 mM) were added at room temperature to a solution of Compound (10) (10.0 g, 30.8 mM) in DMF (150 ml). After the addition, the mixture was stirred at 50° C. for 18 h and then the mixture was diluted with ether (300 ml) and washed with water (3×50 ml). The two layers were separated and the aqueous phase was extracted with dichloromethane (2×50 ml). The organic layers were combined, dried over anhydrous MgSO4 and evaporated under reduced pressure to give an orange oil. The resulting material was purified by silica gel column chromatography (with elution with 10-20% EtOAc/DCM) to obtain Compound (11) (ZE33-0101) as white crystals (10.0 g, 63.4%).

### ZE33-0102 (BDF: T106e)

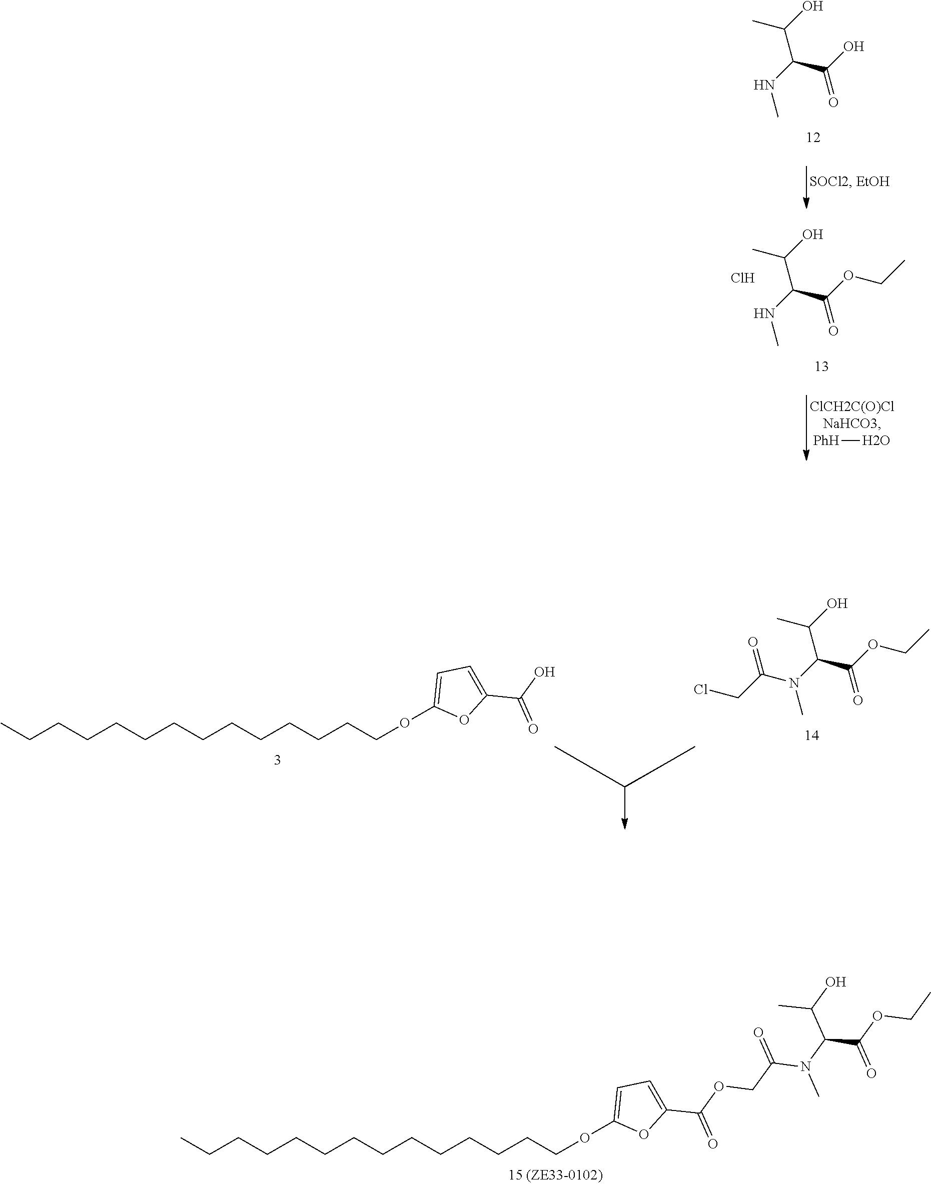

### Compound (13)

SOCl₂ (16.3 g, 136.5 mM) was added dropwise at 0° C. to a solution of Compound (12) (14.0 g, 105.0 mM) in EtOH (200 ml). The resulting mixture was heated for 1 h under reflux cooling. The mixture was subsequently left to stand at room temperature to cool and then concentrated under reduced pressure. After removing the solvent, the residue was poured into aqueous NaHCO₃. The aqueous solution was extracted with DCM, and the extract was washed successively with aqueous NaHCO₃, H₂O, sodium chloride solution and dried over Na₂SO₄. Concentration under reduced pressure gives Compound (13) (20.5 g, 98%) as a colorless powder, which was used directly for the next reaction.

### Compound (14)

Compound (13) (20.5 g, 103.7 mM) was dissolved in 400 ml of water. The solution was cooled using an ice bath and admixed in several steps with (17.4 g, 207.4 mM) NaHCO₃. The reaction mixture was admixed dropwise with a solution (12.9 g, 114.0 mM) of chloroacetyl chloride in 150 ml of toluene, then stirred vigorously at 20-25° C. for 3 h. After phase separation, the aqueous phase was extracted with toluene. The combined organic phases were freed of the solvent to obtain Compound (14) as a colorless oil (20.9 g, 85%).

### Compound 15 (ZE33-0102) (BDF: T106e)

Compound (14) (1.2 eq., 10.5 g, 44.3 mM) and triethylamine (2 eq., 7.5 ml, 73.8 mM) were added mM) at room temperature to a solution of TOFA (3) (12.0 g, 36.9 mM) in DMF (200 ml). After the addition, the mixture was stirred at 50° C. for 18 h and then the mixture was diluted with ether (300 ml) and washed with water (3×50 ml). The two layers were separated and the aqueous phase was extracted with dichloromethane (2×50 ml). The organic layers were combined, dried over anhydrous MgSO₄ and evaporated under reduced pressure to give an orange oil. The resulting material was purified by silica gel column chromatography (with elution with 10-20% EtOAc/DCM) to obtain Compound (15) (ZE33-0102) as white crystals (8.4 g, 43.2%).

### ZE33-0104 (BDF: T107e) and ZE33-0147 (T117e)

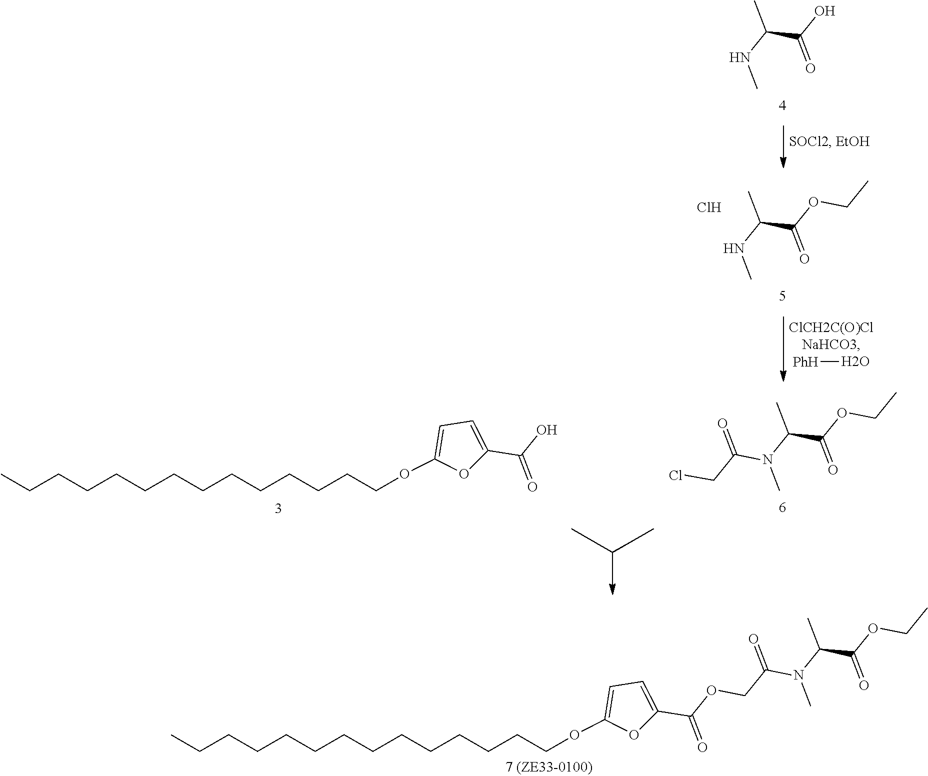

### Compound (16)

HOCH₂CH₂COOt-Bu (9.1 g, 62 mM) was added to a solution of TOFA (3) (14.3 g, 44 mM) in 450 ml of CH₂Cl₂, followed by the addition of EDCI (14.9 g, 77 mM) and DMAP (1.0 g, 8.2 mM). The mixture was stirred at ambient temperature for 20 h, evaporated, dissolved again in Et₂O, washed with H₂O and dried with Na₂SO₄. The residue was purified by column chromatography on silica gel, elution being performed with 5% EtOAc in hexane, which gave

### Compound (16) (12.5 g, 62%).

### Compound (17)

50 ml of TFA was added to a solution of Compound (16) (11.8 g, 26 mM) in 150 ml of CH₂Cl₂ and the mixture was stirred at ambient temperature for 1 hour. The solvents were evaporated and the residue was coevaporated with CCl₄, which gave Compound (17) (10.5 g, 100%).

### Compounds (18) and (19) (ZE33-0104 and ZE33-0147).

Sarcosine ethyl ester hydrochloride (5.6 g, 36.5 mM) was added to a solution of Compound (17) (11.1 g, 28 mM) in 500 ml of CH₂Cl₂, followed by the addition of HOBt (4.2 g, 31 mM), DIPEA (11.6 g, 90 mM) and EDCI (7.6 g, 40 mM). The mixture was stirred at ambient temperature for 20 h, evaporated, dissolved again in EtOAc, washed with H₂O and dried with Na₂SO₄. The residue was purified by column chromatography on silica gel, elution being performed with 0-50% EtOAc in hexane, to produce Compound (18) (ZE33-0104) (7.7 g, 56%) and Compound (19) (ZE33-0147) (3.19 g, 20%).

### ZE33-0105 (BDF: T72e)

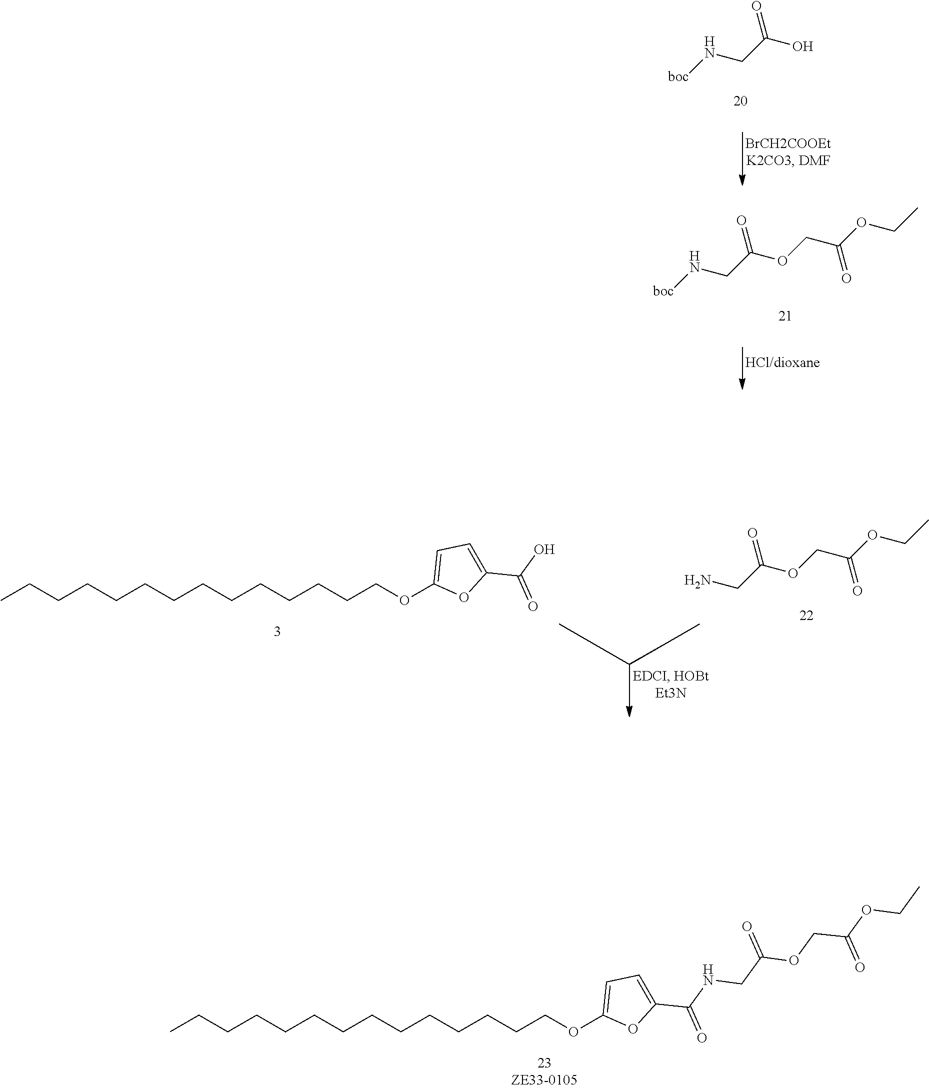

### Compound (21)

Ethyl bromoacetate (8.5 g, 50 mM) was added to a solution of Boc-glycine (20) (8.7 g, 50 mM) in 100 ml of DMF, followed by the addition of K₂CO₃ (20.7 g, 150 mM). The mixture was stirred at 50-60° C. for 15 h, cooled to ambient temperature, diluted with H₂O (400 ml) and extracted with Et₂O. The combined organic extracts were dried with Na₂SO₄ and evaporated, which gave Compound (21) (13.0 g, 99%), which was used for the next step without additional purification.

### Compound (22)

25 ml of TFA was added to a solution of Compound (21) (13.0 g, 5 mM) in 100 ml of CH₂Cl₂ and the mixture was stirred at ambient temperature for 15 hours. The volatile constituents were evaporated off and the residue was basified with aqueous NaHCO₃. The solution was extracted with CH₂Cl₂, the combined organic extracts were dried with Na₂SO₄ and evaporated, which gave Compound (22) (8.0 g, 98%), which was used for the next step without additional purification.

### Compound (23) (ZE33-0105)

EDCI (6.3 g, 32 mM) was added to a solution of Compound (3) (TOFA) (5.3 g, 16 mM) and amine (22) (3.9 g, 24 mM) in 200 ml of CH₂Cl₂, followed by the addition of DMAP (4.4 g, 32 mM) and the mixture was stirred at ambient temperature for 24 h. The mixture was quenched with aqueous citric acid, the organic layer was washed with aqueous NaHCO₃, dried with Na₂SO₄ and evaporated, which gave Compound (23) (ZE33-0105) (6.6 g, 88%).

### ZE33-0107 (BDF: T108e) and ZE33-0119 (T114s)

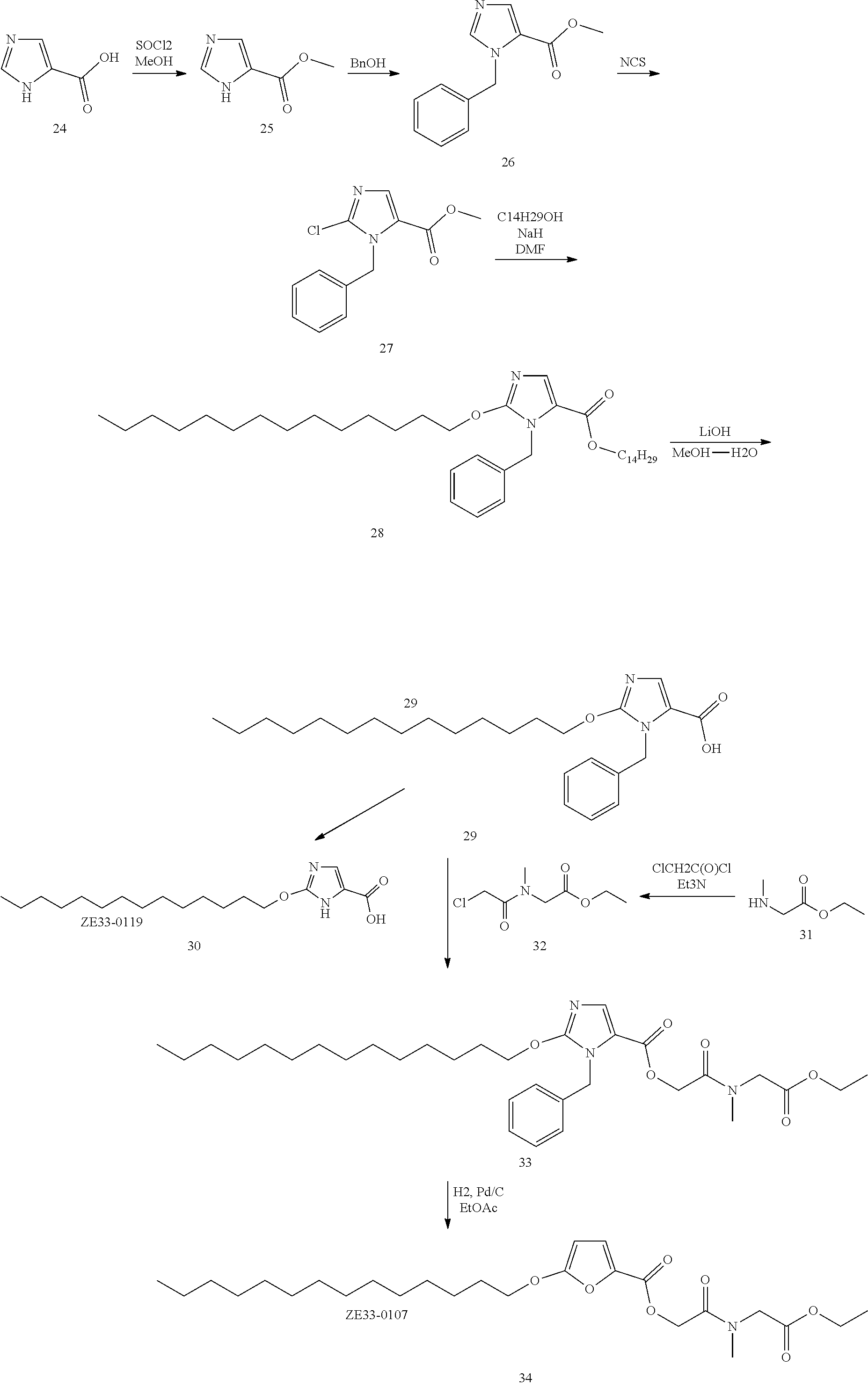

### Compound (25)

SOCl₂ (109.1 g, 918 mM) was added dropwise to a suspension of imidazole-2-carboxylic acid (24) (51.4 g, 459 mM) in 400 ml of MeOH. The mixture was stirred for 15 h under reflux cooling, cooled to ambient temperature and evaporated. The residue was basified with aqueous K₂CO₃, extracted with t-BuOH—CHCl₃, 1:1. The combined extracts were dried with Na₂SO₄, evaporated and coevaporated twice with MeCN, which gave Compound (25) (51.0 g, 88%).

### Compound (26)

Diisopropyl azodicarboxylate (98.1 g, 485 mM) in 100 ml of THF was added dropwise at 5-10° C. to a mixture of Compound (25) (51.0 g, 404 mM), benzyl alcohol (52.4 g, 485 mM) and PPh₃ (127.3 g, 485 mM) in 500 ml of THF. The mixture was stirred at ambient temperature for 15 h and subsequently evaporated. The residue was dissolved in EtOAc and the target compound was extracted with 10% aqueous HCl. The combined aqueous extracts were carefully basified with solid K₂CO₃ at 5-10° C. The mixture was extracted with EtOAc, the combined organic extracts were dried with Na₂SO₄ and evaporated. The residue was purified by column chromatography on silica gel, elution being performed with 25-50% EtOAc in hexane, which gave Compound (26) (68.1 g, 78%).

### Compound (27)

A mixture of Compound (26) (68.1 g, 315 mM) and N-chlorosuccinimide (42.0 g, 315 mM) in 350 ml of DMF was stirred at ambient temperature for 15 hours. The reaction mixture was quenched with aqueous NaHCO₃, extracted with Et₂O, the combined organic extracts were dried with Na₂SO₄ and evaporated. The residue was purified by column chromatography on silica gel, elution being performed with 1-10% EtOAc in hexane. The target fractions were evaporated and the solid residue was washed with hexane, which gave Compound (27) (32.8 g, 42%).

### Compound (28)

NaH (60% in mineral oil, 13.1 g, 328 mM) was added in portions to a suspension of n-tetradecanol (70.1 g, 328 mM) in 600 ml of DMF. The mixture was stirred at 50° C. for 2 h and Compound (27) (32.8 g, 131 mM) was added in solid form. The reaction mixture was stirred at 50° C. for 24 h and then at ambient temperature for a weekend. The mixture was quenched with aqueous NH4Cl, extracted with EtOAc, the combined organic extracts were dried with Na₂SO₄ and evaporated. The residue was purified by column chromatography on silica gel, elution being performed with 1-5% EtOAc in hexane. The target fractions were evaporated and the solid residue was washed with MeOH, which gave Compound (28) (17.8 g, 22%).

### Compound (29)

A mixture of Compound (28) (17.8 g, 29 mM) and LiOH·H₂O (9.8 g, 232 mM) in MeOH—THF—H₂O (300 ml) was stirred at 80° C. for 15 h and then at ambient temperature for 24 h. The organic solvents were evaporated and the aqueous residue was acidified with aqueous KHSO₄.

The mixture was extracted with EtOAc, the combined organic extracts were dried with Na₂SO₄ and evaporated. The solid residue was washed with hexane, which gave Compound (29) (11.3 g, 93%).

### Compound (30) (ZE33-0119).

First a few droplets of concentrated HCl and then 10% Pd/C (30 mg) were added to a solution of Compound (29) (0.31 g, 0.75 mM) in MeOH. The mixture was hydrogenated at 7 MPa for 2 hours at 40° C. The catalyst was filtered off and the solvent was evaporated. The residue was dissolved again in MeOH and basified with Dowex 1×8 chloride form (strongly basic). The mixture was filtered through Celite and evaporated. The residue was suspended in hot MeCN, isolated by filtration and dried, which gave Compound (30) (ZE33-0119) (0.16 g, 67%).

### Compound (32)

Sarcosine ethyl ester hydrochloride (31) (50.00 g, 330 mM) was dissolved in 160 ml of water. The solution was cooled using an ice bath and admixed in several steps with 2.5 eq. (68.4 g, 830 mM) of NaHCO₃. The reaction mixture was mixed dropwise with a solution of (36.7 g, 330 mM) chloroacetyl chloride in 140 ml of toluene, then stirred vigorously at 20-25° C. for 3 h. After phase separation, the aqueous phase was extracted with toluene. The combined organic phases were freed of the solvent. (53.5 g, 84.9% of) Compound (32) was obtained as a colorless oil.

### Compound (33)

A mixture of Compound (29) (11.3 g, 27 mM), chloroacetyl chloride (32) (6.9 g, 35 mM) and Et₃N (16.5 g, 162 mM) in 200 ml of DMF was stirred at 60° C. for 15 h. The mixture was quenched with aqueous NH4Cl, extracted with EtOAc, the combined organic extracts were dried with Na₂SO₄ and evaporated. The residue was purified by column chromatography on silica gel, elution being performed with 20-75% EtOAc in hexane. The target fractions were evaporated and the solid residue was with hexane

### Compound (34) (ZE33-0107)

2.32 g of 10% Pd/C was added to a solution of Compound (33) (11.6 g, 20.3 mM) in 150 ml of EtOAc and the mixture was hydrogenated at 5 MPa and 40-50° C. for 48 hours. The catalyst was filtered off, the filtrate was evaporated and the residue was purified by column chromatography on silica gel, elution being performed with 20-50% EtOAc in hexane 25% CHCl₃ in EtOAc, which gives Compound (34) (ZE33-0107) (5.82 g, 60%).

### ZE33-0110 (T109e) and ZE33-0122

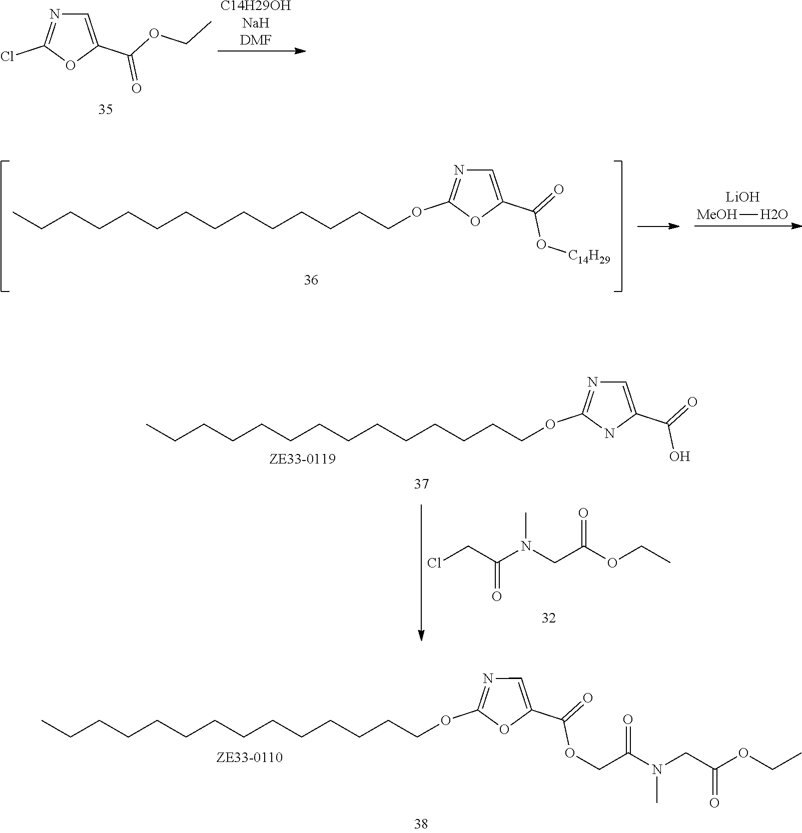

### Compound (37) (ZE33-0122)

60% NaH (1.82 g, 46 mM) was added to a solution of n-tetradecanol (12.3 g, 57.5 mM) in 100 ml of THF. The mixture was stirred at 80° C. for 4 h, cooled to 5° C. and a solution of chlorooxazole (35) (6.08 g, 28.9 mM) in 20 ml of THF was added. The mixture was stirred at ambient temperature for 15 h and quenched with 5% HCl. The mixture was extracted with Et₂O, the combined organic extracts were dried with Na₂SO₄ and evaporated. The residue was washed with hexane and dried, whereby Compound (36) (4.4 g) was obtained, which was used for the next step without additional purification. Compound (36) was dissolved again in 250 ml of THF and 75 ml of MeOH, 1.55 g of LiOH and 5 ml of H₂O were added and the mixture was stirred at ambient temperature for 15 h. The organic constituents were evaporated and the residue was quenched with 1.5% HCl. The pellet formed was washed with H₂O and dried, which gave Compound (37) (ZE33-0122) (2.04 g, 22%).

### Compound (38) (ZE33-0110)

A mixture of Compound (37) (6.1 g, 187 mM), chloride (32) (9.0 g, 46 mM) and Et₃N (9.0 g, 89 mM) in 200 ml of MTBE was stirred at 60° C. for 48 h. The mixture was quenched with aqueous NH4Cl, extracted with EtOAc, the combined organic extracts were dried with Na₂SO₄ and evaporated. The residue was purified by column chromatography on silica gel, elution being performed with 20-50% EtOAc in hexane. The target fractions were evaporated and the solid residue was washed with 10% Et₂O in hexane, which gave Compound (38) (ZE33-0110) (5.0 g. 55%).

### ZE33-0111 (T110e)

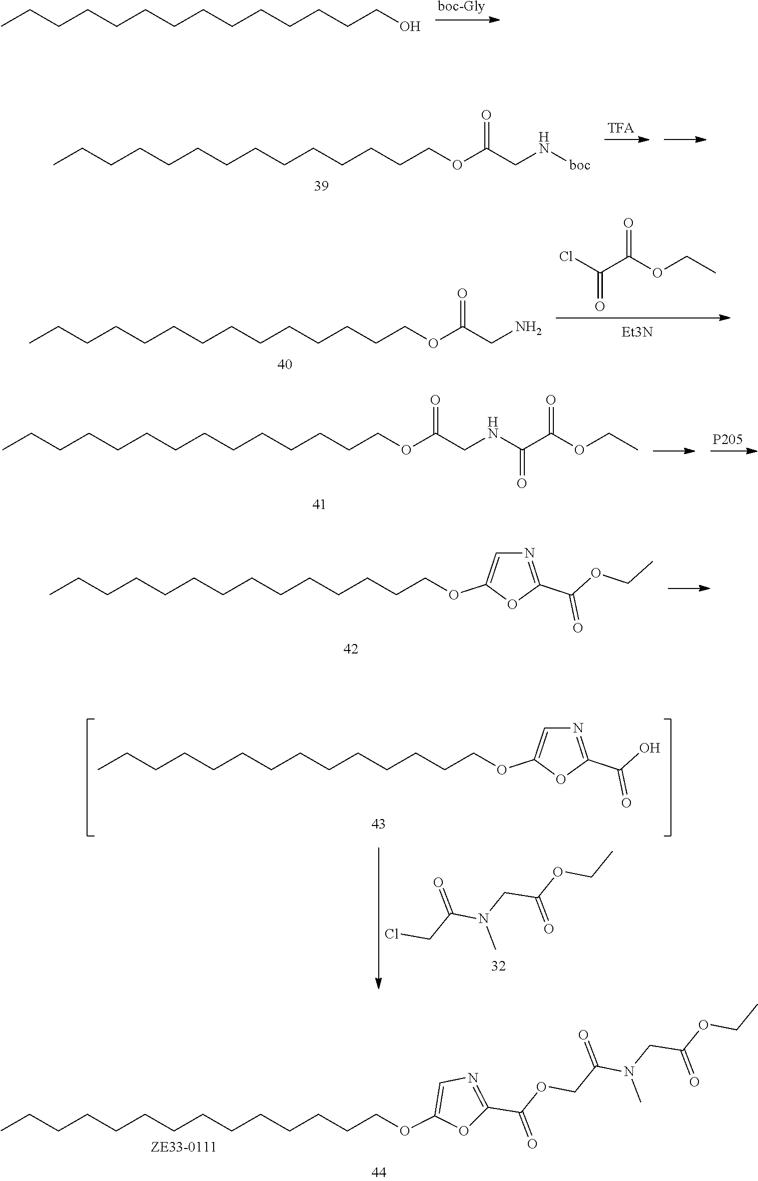

### Compound (39)

EDCI (21.6 g, 112 mM) was added to a solution of n-tetradecanol (20.0 g, 92 mM) in 800 ml of CH₂Cl₂, followed by the addition of DMAP (1.12 g, 0.2 mM) and the mixture was stirred at ambient temperature for 24 hours. The mixture was quenched with 20% aqueous H₂SO₄, the organic layer was washed with aqueous NaHCO₃, dried with Na₂SO₄ and evaporated, which gave Compound (39) (34.8 g, 100%).

### Compound (40)

50 ml of TFA was added to a solution of Compound (39) (34.8 g, 92 mM) in 200 ml of CH₂Cl₂ and the mixture was stirred at ambient temperature for 15 hours. The volatile constituents were evaporated off and the residue was basified with aqueous NaHCO₃. The solution was extracted with CH₂Cl₂, the combined organic extracts were dried with Na₂SO₄ and evaporated, which gave Compound (40) (20.0 g, 80%).

### Compound (41)

Et₃N (22.4 g, 216 mM) was added to a solution of Compound (40) (20.0 g, 72 mM) in CH₂Cl₂ (300 ml), followed by the dropwise addition of ethyl oxalyl chloride (12.0 g, 88 mM) in 40 ml of CH₂Cl₂ at 5° C. The reaction mixture was stirred at ambient temperature for 4 h, quenched with 10% HCl, extracted with CH₂Cl₂, the combined organic extracts were dried with Na₂SO₄ and evaporated. The residue was purified by column chromatography on silica gel, elution being performed with 5-30% EtOAc in hexane, which gave Compound (41) (12.0 g, 43%).

### Compound (42)

A suspension of P₂O₅ (84.4 g, 600 mM) in CH₃CN (150 ml) under nitrogen was heated to 50° C. and treated with Compound (41) (36.8 g, 100 mM) dissolved in 30 ml of CH₃CN. The mixture was heated to 65° C. for 1.5 hours, then cooled in an ice bath. Ice and sodium chloride solution were added to the reaction mixture, then EtOAc was added and the mixture was transferred to a separating funnel. CHCl₃ was added to dissolve solids, and the organic layer was isolated. The aqueous layer was washed repeatedly with CHCl₃ and EtOAc, the organic layers were combined and dried with Na₂SO₄ and then concentrated. The residue was chromatographed on silica gel, elution being performed with a gradient of 0-30% EtOAc/hexane, to give the product as a colorless oil, which was additionally recrystallized with hexane, which gave Compound (42) (27 g, 77%).

### Compound (43)

A mixture of Compound (42) (20.5 g, 60 mM) and LiOH (3.7 g, 90 mM) in a THF/MeOH/H₂O (50 ml, 4 ml, 150 ml) solvent mixture was stirred at 50° C. for 4 hours. The reaction mixture was acidified to pH 2 with aqueous 2 N HCl and the resulting precipitate was isolated by filtration and washed first with H₂O and then with EtOAc to provide Compound (43) as a white solid, which was used for the next step without additional purification (15.4 g, 82%).

### Compound (44) (ZE33-0111)

Compound (32) (11.0 g, 56 mM) and triethylamine (9.5 ml, 94 mM) were added at room temperature to a solution of Compound 43 (15.4 g, 47 mM) in DMF (150 ml). After the addition, the mixture was stirred at 50°° C. for 18 h and then the mixture was diluted with ether (250 ml) and washed with water (2×50 ml). The two layers were separated and the aqueous phase was extracted with dichloromethane (2×50 ml). The organic layers were combined, dried over anhydrous MgSO₄ and evaporated under reduced pressure to give an orange oil. The residue was purified by silica gel column chromatography (with elution with 10-20% EtOAc/DCM) to provide Compound (44) (ZE33-0111) (9.0 g, 39%).

### ZE33-0112 (T111e) and ZE33-0124

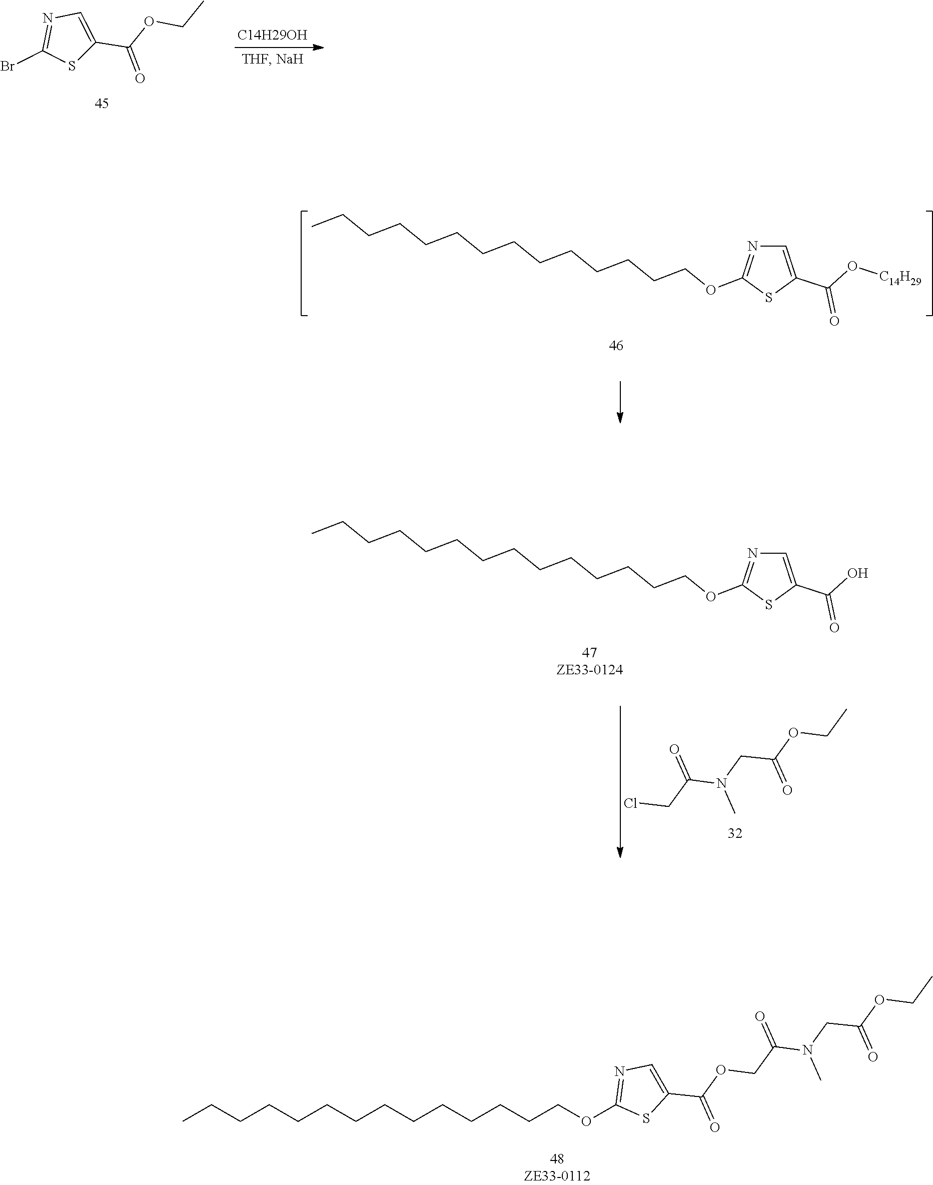

### Compound (47) (ZE33-0124)

NaH (60% in mineral oil) was added in portions (7.4 g, 185 mM) to a suspension of n-tetradecanol (41.7 g, 195 mM) in 200 ml of DMF. The mixture was stirred at 60° C. for 2 h and Compound (45) (23.0 g, 97 mM) was added in 50 ml of DMF. The reaction mixture was stirred at 80° C. for 24 h. The mixture was quenched with 10% aqueous H₂SO₄, extracted with EtOAc, the combined organic extracts were dried with Na₂SO₄ and evaporated. The residue was washed with 10% MTBE in hexane, which gave Compound (47) (ZE33-0124) (7.4 g, 22%).

### Compound (48) (ZE33-0112)

A mixture of Compound (47) (7.4 g, 22 mM), chloride (32) (4.6 g, 24 mM) and Et₃N (6.6 g, 66 mM) in 200 ml of MTBE was stirred at 60° C. for 48 h. The mixture was quenched with aqueous NH₄Cl, extracted with EtOAc, the combined organic extracts were dried with Na₂SO₄ and evaporated. The residue was washed with MeCN—H₂O, 1:1, followed by washing with Et₂O and drying, which gave Compound (48) (8.8 g, 82%).

### ZE33-0113 (T73e) and ZE33-0125

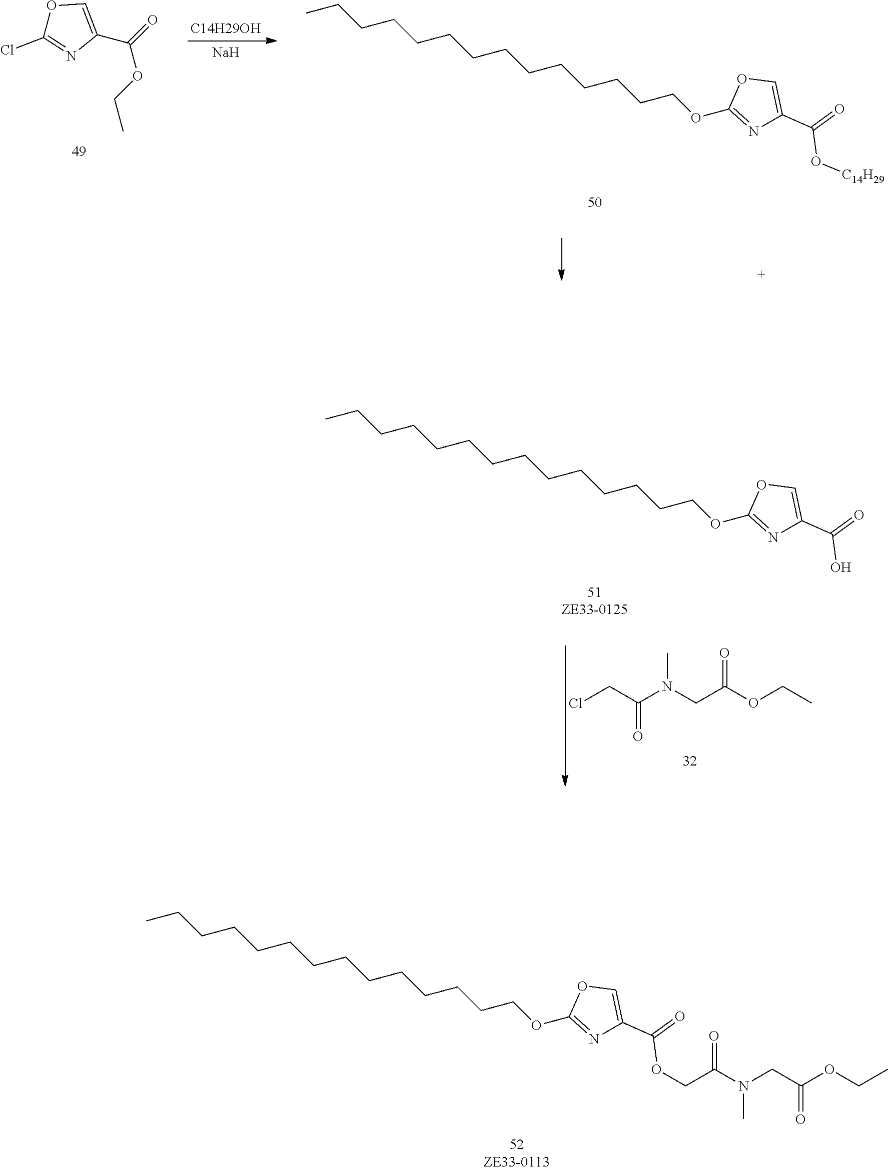

### Compound (50)

60% NaH (8.0 g, 245 mM) was added to a solution of n-tetradecanol (60.0 g, 280 mM) in 500 ml of THF. The mixture was stirred at 80° C. for 4 h, cooled to 5° C. and a solution of chlorooxazole (49) (19.5 g, 111 mM) in 50 ml of THF was added. The mixture was stirred at ambient temperature for 15 h and quenched with 5% HCl. The mixture was extracted with Et₂O, the combined organic extracts were dried with Na₂SO₄ and evaporated. The residue was purified by column chromatography on silica gel (EtOAc-Hx, 0-75%), which gave Compound (50) (20.0 g, 35%).

### Compound (51) (ZE33-0125)

Compound (50) (19.6 g, 38 mM) was dissolved in 250 ml of THF and 75 ml of MeOH, 4.6 g (190 mM) of LiOH and 20 ml of H₂O were added and the mixture was stirred at ambient temperature for 15 h. The organic constituents were evaporated and the residue was quenched with 1.5% HCl. The mixture was extracted with CH₂Cl₂, the combined organic extracts were dried with Na₂SO₄ and evaporated. The residue was purified by column chromatography on silica gel, elution being performed with 0-10% EtOAc in CH₂Cl₂, which gave Compound (51) (ZE33-0125) (19.6 g, 100%).

### Compound (52) (ZE33-0113)

A mixture of Compound (51) (19.5 g, 38 mM), chloride (32) (11.0 g, 57 mM) and Et₃N (17.3 g, 170 mM) in 200 ml of DMF was stirred at 60° C. for 48 h. The mixture was quenched with aqueous NH₄Cl, extracted with EtOAc, the combined organic extracts were dried with Na₂SO₄ and evaporated. The residue was purified by column chromatography on silica gel, elution being performed with 20-50% EtOAc in hexane. The target fractions were evaporated and the solid residue was washed with 10% Et₂O in hexane, which gave Compound (52) (ZE33-0113) (8.77 g, 48%).

### ZE33-0114 (T78e) and ZE33-0126

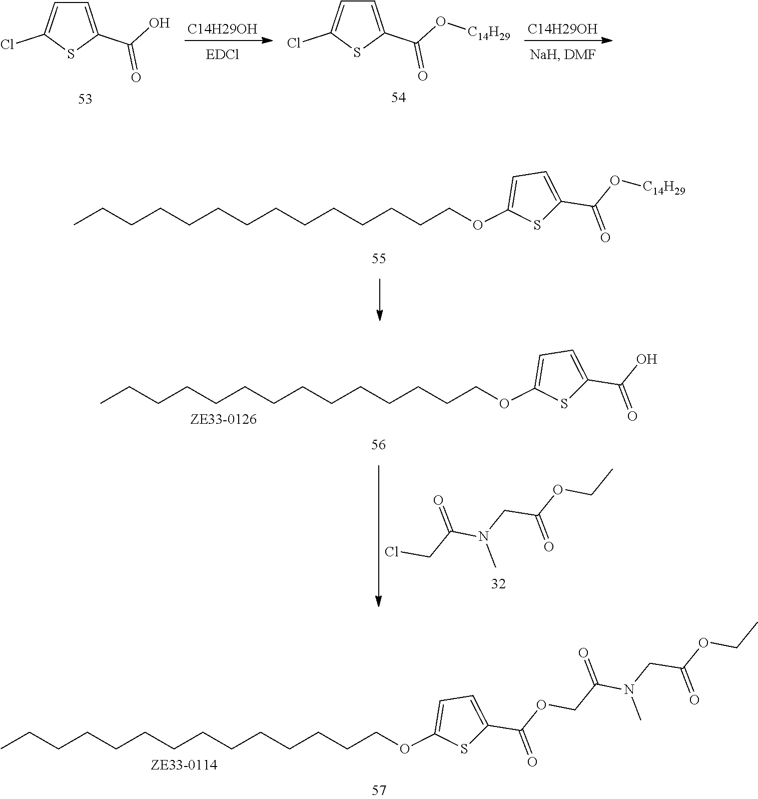

### Compound (54)

EDCI (35.4 g, 185 mM) was added to a solution of chlorothiophenecarboxylic acid (53) (25 g, 154 mM) and n-tetradecanol (39.5 g, 185 mM) in 400 ml of CH₂Cl₂, followed by the addition of DMAP (3.8 g, 31 mM) and the mixture was stirred at ambient temperature for 5 h. The mixture was quenched with 20% aqueous H₂SO₄, the organic layer was dried with Na₂SO₄, passed through an SiO₂ layer and evaporated, which gave Compound (54) (52.8 g, 96%).

### Compound (55)

NaH (60% in mineral oil) was added in portions (11.2 g, 279 mM) to a suspension of n-tetradecanol (63.0 g, 294 mM) in 400 ml of DMF. The mixture was stirred at 60° C. for 3 h and Compound (54) (52.8 g, 147 mM) was added in 100 ml of DMF. The reaction mixture was stirred at 80° C. for 24 h. The mixture was quenched with 10% aqueous H₂SO₄, extracted with EtOAc, the combined organic extracts were dried with Na₂SO₄ and evaporated. The residue was purified by column chromatography on silica gel, elution being performed with 1-5% EtOAc in hexane. The target fractions were evaporated and the solid residue was washed with MeOH, which gave Compound (55) (38.9 g, 49%).

### Compound (56) (ZE33-0126).

50 ml of 10 N aqueous NaOH was added to a solution of Compound (55) (38.9 g, 72 mM) in 1000 ml of EtOH-H₂O, 1:1, and the mixture was stirred at 120° C. for 15 h. MeOH was evaporated, Et₂O was added and the middle layer that contained the target compound was removed and acidified with 10% aqueous H₂SO₄. The combined ether extracts were dried with Na₂SO₄ and evaporated, the solid residue was washed with hexane and dried, which gave Compound (56) (ZE33-0126) (14.3 g, 58%).

### Compound (57) (ZE33-0114)

A mixture of Compound (56) (7.2 g, 22 mM), chloride (32) (4.0 g, 22 mM) and Et₃N (4.2 g, 44 mM) in 200 ml of DMF was stirred at 60° C. for 15 h. The mixture was quenched with aqueous NH₄Cl, extracted with EtOAc, the combined organic extracts were dried with Na₂SO₄ and evaporated. The residue was purified by column chromatography on silica gel, elution being performed with 20-30% EtOAc in hexane. The target fractions were evaporated and the solid residue was washed with 10% Et₂O in hexane, which gave Compound (57) (ZE33-0114) (7.38 g, 70%).

### ZE33-0115 (T112e) and ZE33-0127

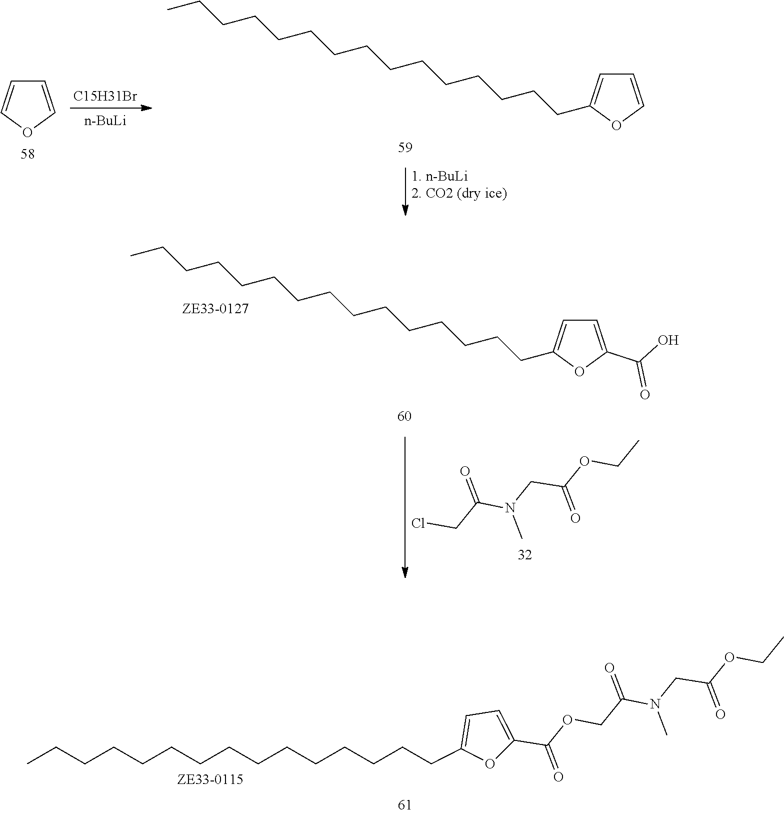

### Compound (59)

30 ml of 2.5 M n-BuLi in hexane was added dropwise at −70° C. to a solution of furan (58) (4.9 g, 72 mM) in 30 ml of THF. The mixture was stirred at −70° C. for 0.5 h and 0° C. for 1 h, cooled to −70° C. and admixed with a solution of n-pentadecyl bromide (18.0 g, 62 mM) in 20 ml of THF. The mixture was stirred at ambient temperature for 15 h, quenched with aqueous NH₄Cl, extracted with Et₂O, the combined organic extracts were dried with Na₂SO₄ and evaporated. The residue was purified by column chromatography on silica gel with elution with hexane, which resulted in Compound (59) (11.9 g, 69%).

### Compound (60) (ZE33-0127)

20 ml of 2.5 M n-BuLi in hexane was added dropwise at −30° C. to a solution of Compound (59) (9.7 g, 35 mM) in 100 ml of THF. The mixture was stirred at ambient temperature for 3.5 h, cooled to −35° C. and admixed with freshly crushed dry ice (˜400 g). The mixture was stirred at ambient temperature for 15 h and quenched with aqueous KHSO₄. The mixture was extracted with CH₂Cl₂, the combined organic extracts were dried with Na₂SO₄ and evaporated. The solid residue was washed with hexane, which gave Compound (60) (ZE33-0127) (9.5 g, 91%).

Compound (61) (ZE33-0115)

A mixture of Compound (60) (11.6 g, 36 mM), chloride (32) (11.7 g, 60 mM) and Et₃N (35 g, 35 mM) in 150 ml of DMF was stirred at 60° C. for 24 h. The mixture was quenched with aqueous NH₄Cl, extracted with EtOAc, the combined organic extracts were dried with Na₂SO₄ and evaporated. The residue was purified by column chromatography on silica gel, elution being performed with 0±5% EtOAc in CH₂Cl₂, which gave Compound (61) (ZE33-0115) (13.2 g, 76%).

### ZE33-0116 (T113e)

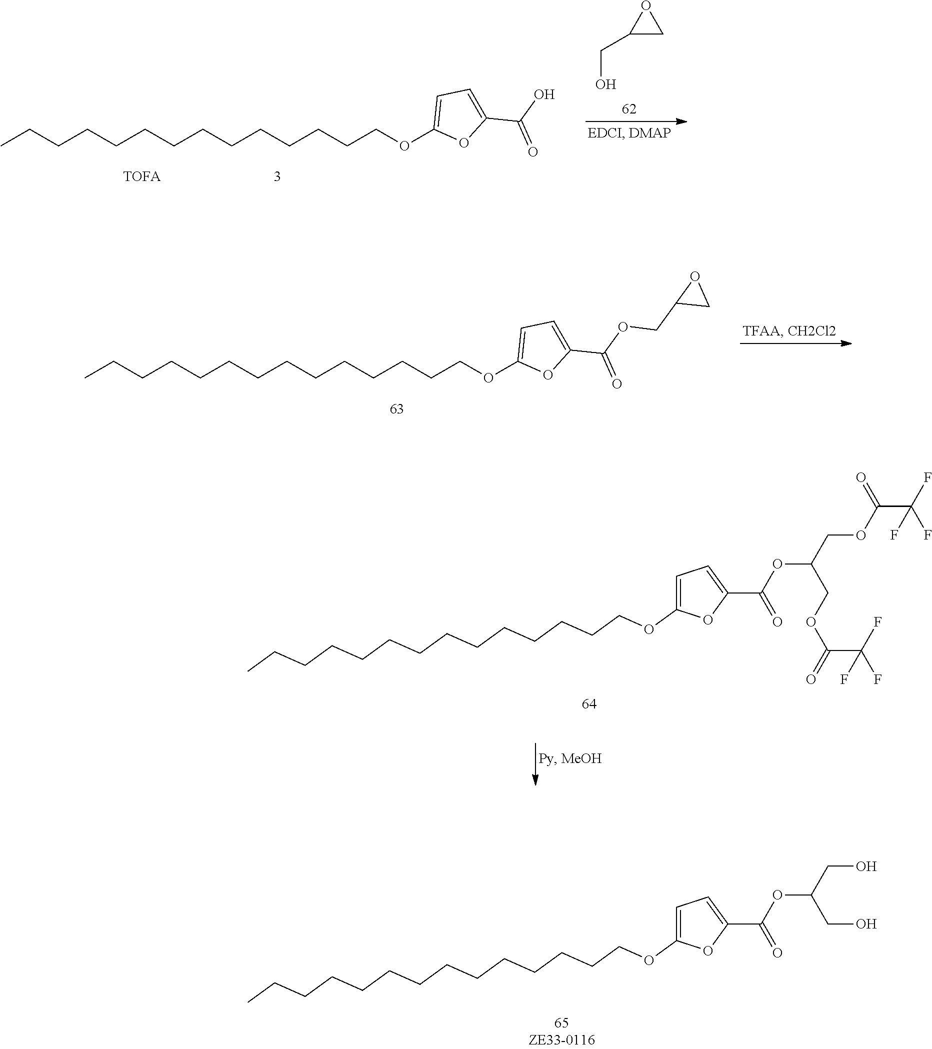

### Compound (63)

Glycidol (62) (3.78 g, 51 mM) was added to a solution of TOFA (3) (13.1 g, 40 mM) in 500 ml of CH₂Cl₂, followed by the addition of EDCI (10.5 g, 55 mM) and DMAP (0.97 g, 8 mM) and the mixture was stirred at ambient temperature for 20 h. The solvent was evaporated, the residue was dissolved again in EtOAc, washed with H₂O and dried over Na₂SO₄ and evaporated. The residue was purified by column chromatography on silica gel, elution being performed with 15% EtOAc in hexane, which gave Compound (63) (9.43 g, 61%).

### Compound (64)

Trifluoroacetic anhydride (26.4 g, 126 mM) in 40 ml of CH₂Cl₂ was added at −30° C. to a solution of 9.43 g (25 mM) of Compound (63) in 120 ml of CH₂Cl₂. The mixture was stirred at ambient temperature for 4 hours and evaporated, the residue was coevaporated with CCl₄, which gave Compound (64) (14.5 g, 99%).

### Compound (65) (ZE33-0116)

A solution of pyridine (22 g) and MeOH (13 g) in 100 ml of CH₂Cl₂ was added dropwise at −55° C. to a solution of Compound (64) (14.5 g, 25 mM) in 120 ml of CH₂Cl₂. The mixture was stirred at −30° C. for 0.5 h and then at ambient temperature for 3 h. The mixture was evaporated and the residue was washed with hexane, which gave Compound (65) (ZE33-0116) (9.85 g, 100%).

### ZE33-0117 (T74e)

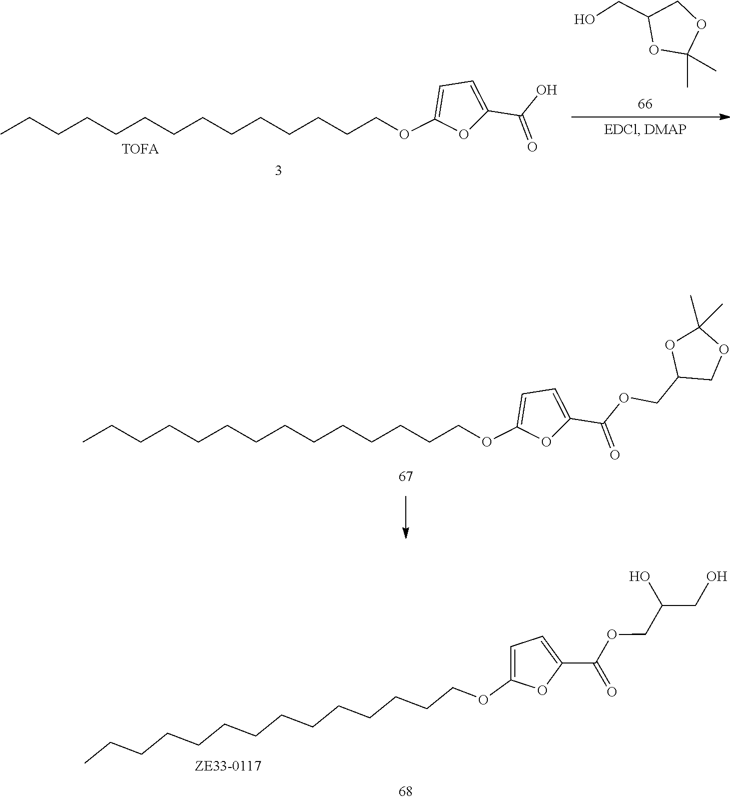

### Compound (67)

Protected glycerol (66) (4.1 g, 31 mM) was added to a solution of TOFA (3) (7.9 g, 24 mM) in 500 ml of CH₂Cl_{2,} followed by the addition of EDCI (6.3 g, 33 mM) and DMAP (0.61 g, 5 mM) and the mixture was stirred at ambient temperature for 20 h. The solvent was evaporated, the residue was dissolved again in EtOAc, washed with H₂O and dried over Na₂SO₄ and evaporated. The residue was purified by column chromatography on silica gel, elution being performed with 15% EtOAc in hexane, which gave Compound (67) (9.16 g, 86%).

### Compound (68) (ZE33-0117)

0.4 ml of 3 M HCl in dioxane was added to a solution of Compound (67) (8.78 g, 20 mM) in 400 ml of MeOH and the mixture was stirred at ambient temperature for 0.5 hours. The mixture was evaporated and subsequently coevaporated with MeOH to a volume of approx. 20 ml, the pellet formed was isolated by filtration and dried, which gave Compound (68) (ZE33-0117) (6.9 g, 87%).

The compounds according to the invention can advantageously be incorporated into cosmetic or dermatological preparations.

The preparations or uses according to the invention may be in the form of liquid compositions that can be applied using brushes or spreaders, roll-on devices or atomizers, as sticks and in the form of systems that can be applied from normal bottles and containers, for example creams, gels or lotions. For example, they may thus constitute a solution, an emulsion of water-in-oil (W/O) type or of oil-in-water (O/W) type, or a multiple emulsion, for example of water-in-oil-in-water (W/O/W) type, oil-in-water-in-oil (O/W/O) type, a gel, a hydrodispersion, a lamellar phase, a liquid isotropic solution phase, a micellar phase, a solid or dispersed mono- or polyhexagonal phase, a solid or dispersed mono-or polycubic phase, a lyotropic phase, a crystalline phase, a solid stick or else an aerosol.

Furthermore, the preparations or uses according to the invention may advantageously be in the form of facial toners, tinctures, cleansing formulations, pads, cotton balls or wipes, and in the form of tonics or shampoos.

Advantageously, the pH of the preparations or uses according to the invention is set in the weakly acidic to neutral range, preferably from 3.0-7.0, particularly preferably from 4.0-6.5.

The formulations according to the invention may have the customary composition and be used for the treatment of the skin and/or the hair in the sense of a dermatological treatment or a treatment in the sense of care cosmetics. They may alternatively be used in makeup products in decorative cosmetics or in the cosmetic and dermatological cleansing products.

It is of course known to those skilled in the art that cosmetic preparations are usually not conceivable without the customary auxiliaries and additives. The cosmetic and dermatological preparations according to the invention may accordingly further comprise cosmetic auxiliaries, such as those usually used in such preparations; for example consistency regulators, preservatives, stabilizers, fillers, perfumes, pigments that have a coloring effect, thickeners, suspending agents, buffer mixtures, surface-active substances, emulsifiers, softening, moistening and/or moisturizing substances, anti-inflammatory substances, additional active ingredients such as vitamins or proteins, light stabilizers, insect repellents, bactericides, water, salts, antimicrobial, proteolytic or keratolytic substances, medicaments or other customary constituents of a cosmetic or dermatological formulation such as alcohols, polyols, polymers, foam stabilizers, organic solvents or else electrolytes.

The amounts of carrier substances to be used in each case can easily be determined by those skilled in the art by simple trial and error, depending on the type of the product in question.

As customary carrier substances for the production of the preparations or uses according to the invention, it is possible to use not only water, ethanol and isopropanol, glycerol and propylene glycol, but also skincare lipids or lipoids, such as decyl oleate, cetyl alcohol, cetylstearyl alcohol and 2-octyldodecanol, in the proportions customary for such preparations, and mucilaginous substances and thickeners, for example hydroxyethylcellulose or hydroxypropylcellulose, polyacrylic acid, polyvinylpyrrolidone, but additionally also, in small amounts, cyclic silicone oils (polydimethylsiloxanes) and liquid polymethylphenylsiloxanes of low viscosity.

Emulsifiers that can be used in the preparations in a small amount, for example 1% to 6% by weight, based on the total composition, and that have proved to be suitable for the production of the preparations or uses according to the invention, which are advantageously to be applied as liquid or solid preparations to the desired skin regions, are noniogenic types, such as polyoxyethylene fatty alcohol ethers, for example cetostearyl alcohol polyethylene glycol ether with 12 or 20 added-on ethylene oxide units per molecule, cetostearyl alcohol, and sorbitan esters and sorbitan ester-ethylene oxide compounds (for example sorbitan monostearate and polyoxyethylene sorbitan monostearate), and long-chain higher molecular weight waxy polyglycol ethers. However, a whole range of other emulsifiers or emulsifier mixtures which are usually used in cosmetic preparations are additionally also suitable. These include for example, but are not restricted to, glyceryl stearate citrate, PEG-40 stearate or else polyglyceryl-3 methylglucose distearate, stearic acid, steareth-2 and steareth-21.

The oil phase of the preparations according to the invention is advantageously selected from the group of esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, from the group of esters of aromatic carboxylic acids and saturated and/or unsaturated, branched and/or unbranched alcohols having a chain length of 3 to 30 carbon atoms.

In addition, the oil phase may advantageously be selected from the group of branched and unbranched hydrocarbons and hydrocarbon waxes, silicone oils, dialkyl ethers, the group of saturated or unsaturated, branched or unbranched alcohols, and fatty acid triglycerides, namely the triglyceryl esters of saturated and/or unsaturated, branched and/or unbranched alkanecarboxylic acids having a chain length of 8 to 24, in particular 12-18, carbon atoms. The compounds of the oil phase may be of synthetic, semi-synthetic or natural origin.

Any blends of such oil and wax components may also advantageously be used in the context of the present invention.

The content of the oil phase is advantageously between 1% and 50% by weight, based on the total weight of the preparations, preferably 2%-30% by weight, especially preferably below 10% by weight.

The cosmetic and/or dermatological formulations according to the invention are used by applying a sufficient amount to the skin, scalp and/or the hair in the manner customary for cosmetics and dermatological products.

### Lipid Assay for Testing the Effectiveness of TOFA Derivatives According to the Invention

To determine the lipid-reducing effect of active ingredients, primary cell lines from sebaceous glands of different human donors are used. The cells are cultured for a total of 7 days. After the cells are seeded on day 1, the active ingredient treatment is effected on day 3 and day 5. On day 7, the cells are stained with the dye AdipoRed. This makes it possible to fluorescently stain and to quantify the lipid droplets inside the cells. The dye fluorescein diacetate (FDA), which is converted by living cells, is also used to monitor the vitality of the cells. The resulting fluorescence signal can likewise be quantified. The effect of an active ingredient can be measured and presented as the ratio of the AdipoRed and FDA fluorescence signal relative to the ratio of the corresponding solvent. The tested substances were dissolved in dimethyl sulfoxide (DMSO); all results were accordingly normalized to the DMSO-treated control.

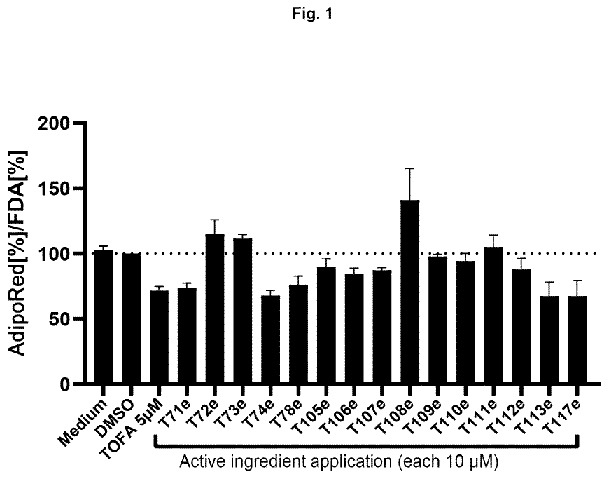

FIG. 1 shows the results of the assay.

The examples that follow are intended to elucidate, but not restrict, the invention. Unless stated otherwise, the figures are based on % by weight.

| | | | | Example |
|---|---|---|---|---|
| | Example 1 | Example 2 | Example 3 | 4 |
| Caprylic/Capric Triglyceride | | | | |
| Propylene Glycol + Aqua | | | 5.00 | 5.00 |
| Citric Acid | | 0.00 | 0.00 | 0.00 |
| Cetearyl Alcohol | 2.00 | | | |
| Aqua + Trisodium EDTA | 1.00 | | | |
| Phenoxyethanol + Aqua | 0.40 | | | |
| Dimethicone | 3.00 | | | |
| Glycerol | 6.00 | 4.00 | 4.00 | 10.00 |
| PEG-40 Hydrogenated Castor Oil | | | 1.00 | 5.00 |
| Butylene Glycol + Aqua | | 20.00 | 10.00 | 10.00 |
| Hydroxyethylcellulose + Disodium | 0.15 | | | |
| Phosphate + Sodium Phosphate | | | | |
| Alcohol Denat. + Aqua | 8.00 | 50.00 | 40.00 | 40.00 |
| Xanthan Gum | 0.15 | | | |
| Acrylates/C10-30 Alkyl Acrylate | 0.15 | | | |
| Crosspolymer | | | | |
| PEG-150 Distearate | 0.50 | | | |
| Tapioca Starch + Aqua | 1.00 | | | |
| Ammonium Acryloyldimethyltaurate/VP | 0.50 | 0.65 | 0.65 | 0.65 |
| Copolymer + Aqua | | | | |
| Methylpropanediol | | | 4.00 | 4.00 |
| Sodium Stearoyl Glutamate + Sodium | 0.30 | | | |
| Chloride | | | | |
| TOFA derivative T105e | 2.00 | | | |
| TOFA derivative T71e | | 1.00 | | |
| TOFA derivative T74e | | | 2.00 | |
| TOFA derivative T113e | | | | 2 |
| Water to | 100.00 | 100.00 | 100.00 | 100.00 |

| | Example 5 | Example 6 | Example 7 |
|---|---|---|---|
| Caprylic/Capric Triglyceride | | | 4.00 |
| Propylene Glycol + Aqua | 5.00 | | |
| Citric Acid | 0.00 | | |
| Cetyl Alcohol | | | 3.00 |
| Aqua + Trisodium EDTA | | 1.00 | 1.00 |
| Diisopropyl Adipate | | 10.00 | 10.00 |
| Phenoxyethanol + Aqua | | 0.50 | 0.80 |
| Hydrogenated Coco-Glycerides | | | 2.00 |
| Glycerol | 10.00 | 3.00 | 9.00 |
| Aqua + Sodium Hydroxide | | 0.06 | 0.01 |
| PEG-40 Hydrogenated Castor Oil | 5.00 | | |
| Butylene Glycol + Aqua | 10.00 | | 5.00 |
| Alcohol Denat. + Aqua | 40.00 | 10.00 | 5.00 |
| Xanthan Gum | | 0.30 | 0.30 |
| Acrylates/C10-30 Alkyl Acrylate | | | 0.20 |
| Crosspolymer | | | |
| Glyceryl Stearate Citrate | | | 2.00 |
| Acrylates/C10-30 Alkyl Acrylate | | 0.20 | |
| Crosspolymer | | | |
| Ammonium Acryloyldimethyltaurate/ | 0.65 | | |
| VP | | | |
| Copolymer + Aqua | | | |
| Methylpropanediol | 4.00 | | |
| TOFA derivative T105e | 0.50 | | |
| TOFA derivative T113e | | 0.50 | |
| TOFA derivative T117e | | | 1 |
| Water to | 100.00 | 100.00 | 100.00 |

1.-6. (canceled)

7. A compound of general formula (1): 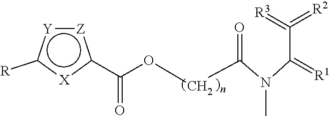
in which
R=n-tetradecyl,
n=a number from 1 to 5,
X=O, N, N—H or S,
Y and Z independently are C—H, N, N—H, O or S, Y and Z being selected such that
the five-membered ring in the above formula has Hückel-aromatic character,
that a double bond cannot simultaneously originate from R² and R³,
and R¹, R² and R³ independently represent —H, an oxo group, —C₁-C₂₄ alkyl (linear and branched), —C₁-C₂₄ alkenyl (linear and branched), —C₁-C₈ cycloalkyl, —C₁-C₈ cycloalkyl-alkylhydroxy, —C₁-C₂₄ alkyl (linear and branched) substituted by one or more hydroxyl groups and/or by one or more primary, secondary and/or tertiary amino groups and/or —C₁-C₂₄ alkyl (linear and branched) substituted with one or more optionally esterified carboxyl groups, —C₁-C₂₄ alkylaryl-alkylhydroxy (linear and branched), —C₁-C₂₄ alkylheteroaryl (linear and branched), —C₁-C₂₄ alkyl-O—C₁-C₂₄ alkyl (linear and branched), —C₁-C₂₄ alkylmorpholino, —C₁-C₂₄ alkylpiperidino, —C₁-C₂₄ alkylpiperazino, —C₁-C₂₄ alkylpiperazino-N-alkyl, oxo groups (═O) provided that at most one of the groups R¹, R² and R³ represents a H atom.

8. The compound of claim 7, the compound being selected from:

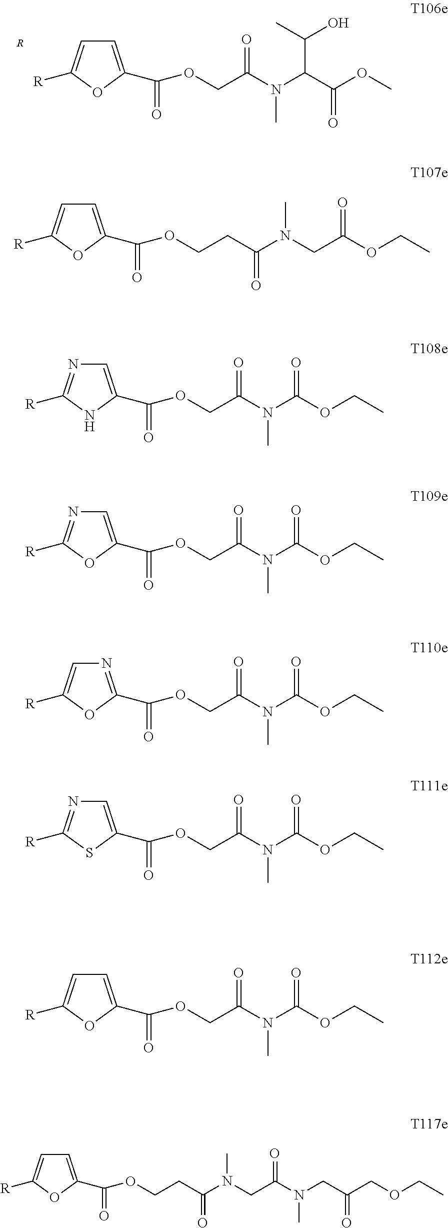

9. A medicinal or cosmetic topical preparation, wherein the preparation comprises at least one compound according to claim 7.

10. A medicinal or cosmetic topical preparation, wherein the preparation comprises at least one compound according to claim 8.

11. The preparation of claim 9, wherein the at least one compound is present in a concentration of from 0.0001% to 40.00% by weight, based on a total weight of the preparation.

12. The preparation of claim 9, wherein the at least one compound is present in a concentration of from 0.001% to 20.00% by weight, based on a total weight of the preparation.

13. The preparation of claim 9, wherein the at least one compound is present in a concentration of from 0.005% to 6.00% by weight, based on a total weight of the preparation.

14. The preparation of claim 10, wherein the at least one compound is present in a concentration of from 0.0001% to 40.00% by weight, based on a total weight of the preparation.

15. The preparation of claim 10, wherein the at least one compound is present in a concentration of from 0.001% to 20.00% by weight, based on a total weight of the preparation.

16. The preparation of claim 10, wherein the at least one compound is present in a concentration of from 0.005% to 6.00% by weight, based on a total weight of the preparation.

17. A method of reducing sebum production in human skin, wherein the method comprises applying to human skin in need thereof an effective amount of the preparation of claim 9.

18. A method of treating greasy and/or impure human skin, wherein the method comprises applying to human skin in need thereof an effective amount of the preparation of claim 9.

19. A method of treating a mild form of acne, wherein the method comprises applying to human skin in need thereof an effective amount of the preparation of claim 9.

20. A method of treating greasy hair and/or a greasy scalp, wherein the method comprises applying to hair or a scalp in need thereof an effective amount of the preparation of claim 9.

21. A method of treating seborrhea, wherein the method comprises applying to human skin in need thereof an effective amount of the preparation of claim 9.
